(19) United States
(12) Patent Application Publication
(10) Pub. No.: US 2024/0262906 A1
(43) Pub. Date: Aug. 8, 2024

(54) ANTI-IGSF1 ANTIBODY AND USE THEREOF

(71) Applicant: WELLMARKER BIO CO., LTD., Seoul (KR)

(72) Inventors: Seong-Rak KIM, Seoul (KR); Hye-Jin SON, Seoul (KR); Mi-So LEE, Seoul (KR); Ha-Na KIM, Seoul (KR); Jun-Hyung LEE, Seoul (KR); Won-Hwa SHIN, Seoul (KR)

(21) Appl. No.: 17/997,019

(22) PCT Filed: Aug. 20, 2021
(86) PCT No.: PCT/KR2021/011139

§ 371 Date: Oct. 24, 2022

(51) Int. Cl. C07K 16/28; A61K 39/00; A61P 35/00

(52) U.S. Cl. CPC C07K 16/2803; A61P 35/00; A61K 2039/505; C07K 2317/565

(57) ABSTRACT

The present invention relates to a novel antibody that specifically binds to IGSF1 and a pharmaceutical composition for preventing or treating cancer, comprising the same as an active ingredient. Specifically, the present invention provides an antibody that binds to the C-terminus of IGSF1. The anti-IGSF1 antibody according to the present invention exhibited high specificity and high binding capacity to IGSF1. The anti-IGSF1 antibody according to the present invention increased the infiltration of immune cells in the spheroids when lung cancer cell spheroids in which IGSF1 is overexpressed were co-cultured with human peripheral mononuclear cells. In addition, the anti-IGSF1 antibody according to the present invention inhibited tumor growth in a humanized mouse transplanted with human lung cancer cells in which IGSF1 is overexpressed, and increased the expression of cytokines in tumor tissues. Through the above results, it was confirmed that the anti-IGSF1 antibody may inhibit tumor growth by increasing the infiltration of immune cells into lung cancer tissues in which the IGSF1 expression is increased, and immune response. Therefore, the anti-IGSF1 antibody may be utilized as an anticancer agent for effectively treating cancer in which IGSF1 is overexpressed.

### TECHNICAL FIELD

The present invention relates to a novel antibody that specifically binds to IGSF1 and a pharmaceutical composition for preventing or treating cancer, comprising the same as an active ingredient.

### BACKGROUND ART

Although research on cancer has been conducted in depth for a long time, the incidence of cancer continues to increase due to environmental pollution and bad eating habits. More than 100 million cancer patients occur worldwide every year, and the World Health Organization (WHO) ranks cancer as one of the leading causes of death. As such, cancer is a major disease that occupies the first place in mortality in modern society, and there is no epoch-making treatment despite many studies to date.

In the treatment of cancer, chemotherapies such as anticancer agents are effective to some extent, but many studies are required due to various pathogenesis of cancer and resistance to anticancer agents. Although the cancer treatment rate has improved due to the development of diagnosis and treatment techniques in recent decades, the 5-year survival rate for many advanced cancers remains in the range of 5 to 50%. In addition, in some cancers, despite various studies and treatments, the survival rate over the past 20 years has not changed significantly.

As such, cancer is not easily treated by conventional cancer treatment regimens, relapses, and metastases to other sites occur, so a more essential treatment method is required. Accordingly, there is growing interest in developing substances for treating cancer by targeting biomarkers that are characteristic of cancer cells that are determined to be the cause of malignancy, metastasis, and recurrence of cancer.

On the other hand, Korean Patent Application Publication No. 2016-0014564 discloses that the IGSF1 (immunoglobulin superfamily member 1) gene may be used as a biomarker for predicting sensitivity to MET (mesenchymal-epithelial transition factor) inhibitors. In the above literature, it is disclosed that an anticancer agent having a high therapeutic effect may be selected by determining the sensitivity of each patient using the biomarker before treating a patient with cancer. However, it has not been disclosed that an antibody specific for IGSF1 may be utilized as an anticancer agent.

### DETAILED DESCRIPTION OF INVENTION

### Technical Problem

The present inventors have studied to develop an anticancer agent having a high therapeutic effect, in particular, a therapeutic agent for effectively treating a cancer in which IGSF1 is overexpressed. As a result, the present inventors have developed an antibody that specifically binds to the C-terminus of IGSF1, and found that the antibody effectively prevents or treats cancer.

### Solution to Problem

In order to achieve the above object, in one aspect of the present invention, there is provided an anticancer agent comprising an anti-IGSF1 antibody that specifically binds to the C-terminus of IGSF1 as an active ingredient.

In another aspect of the present invention, there is provided an antibody specific for IGSF1 or a fragment thereof, comprising a heavy chain variable region comprising H-CDR1 of SEQ ID NO: 1, H-CDR2 of SEQ ID NO: 2 and H-CDR3 of SEQ ID NO: 3; and a light chain variable region comprising L-CDR1 of SEQ ID NO: 4, L-CDR2 of SEQ ID NO: 5 and L-CDR3 of SEQ ID NO: 6.

In another aspect of the present invention, there is provided a polynucleotide encoding the antibody specific for IGSF1 or fragment thereof, an expression vector comprising the polynucleotide, and a transformed cell into which the expression vector is introduced.

In another aspect of the present invention, there is provided a method of producing an antibody specific for IGSF1 or a fragment thereof, comprising culturing the transformed cell; and recovering an anti-IGSF1 antibody or a fragment thereof.

In another aspect of the present invention, there is provided a pharmaceutical composition for preventing or treating cancer, comprising the antibody specific for IGSF1 or fragment thereof as an active ingredient.

In another aspect of the present invention, there is provided a method for preventing or treating cancer, comprising administering to a subject the antibody specific for IGSF1 or fragment thereof.

### Effects of Invention

The anti-IGSF1 antibody according to the present invention exhibited high specificity and high binding capacity to IGSF1. The anti-IGSF1 antibody according to the present invention increased the infiltration of immune cells in the spheroids when lung cancer cell spheroids in which IGSF1 is overexpressed were co-cultured with human peripheral mononuclear cells. In addition, the anti-IGSF1 antibody according to the present invention inhibited tumor growth in a humanized mouse transplanted with human lung cancer cells in which IGSF1 is overexpressed. In addition, the antibody increased the expression of cytokines in cytotoxic T lymphocytes present in tumor tissues. Through the above results, it was confirmed that the anti-IGSF1 antibody may inhibit tumor growth by increasing the infiltration of immune cells into tumor tissues in which the IGSF1 expression is increased and immune response. Therefore, the anti-IGSF1 antibody may be utilized as an anticancer agent for effectively treating cancer in which IGSF1 is overexpressed.

### BRIEF DESCRIPTION OF DRAWINGS

FIG. 1 illustrates a result obtained by confirming the expression level of IGSF1 in IGSF1 overexpressing human lung cancer cells (NCI-H292 IGSF1 O/E) and the control (NCI-H292 MOCK) through Western blot and RT-PCR.

FIG. 2 illustrates a result obtained by confirming tumor-infiltrating lymphocytes (TIL) present in the spheroids when IGSF1 overexpressing human lung cancer cells (NCI-H292 IGSF1 O/E) and the control (NCI-H292 MOCK) spheroids were co-cultured with human peripheral mononuclear cells (PBMC).

FIG. 3 is a graph showing a result obtained by analyzing through flow cytometry the distribution degree of hCD45+ cells in order to confirm the presence of tumor-infiltrating lymphocytes in the tumor tissues of mice transplanted with IGSF1 overexpressing human lung cancer cells (NCI-H292 IGSF1 O/E) or the control (NCI-H292 MOCK).

FIG. 4 illustrates a result obtained by confirming by immunohistochemistry staining method the expression of IGSF1 and the presence of tumor-infiltrating lymphocytes in the tumor tissues of mice transplanted with IGSF1 overexpressing human lung cancer cells (NCI-H292 IGSF1 O/E) or the control (NCI-H292 MOCK).

FIG. 5 is a graph showing a result obtained by analyzing the binding affinity of the WM-A1-3389 antibody to the IGSF1 antigen using ELISA.

FIG. 6 is a graph showing a result obtained by analyzing the binding affinity of the WM-A1-3389 antibody to the IGSF1 antigen in cells using FACS analysis.

FIG. 7 is a graph showing a result obtained by analyzing the binding capacity of the WM-A1-3389 antibody to IGSF1 expressed in cells in IGSF overexpressing human lung cancer cells (NCI-H292 IGSF1 O/E) and the control (NCI-H292 MOCK) using FACS analysis.

FIG. 8 is a graph showing a result obtained by analyzing the binding specificity of the WM-A1-3389 antibody in IGSF1 knock-down (K/D) cell line treated with shIGSF1 in two IGSF1 overexpressing human lung cancer cells (NCI-H292 IGSF1 O/E and HEK293E IGSF1 O/E) and the control (NCI-H292 MOCK and HEK293E MOCK).

FIG. 9 illustrates a result obtained by confirming through a microscope image tumor-infiltrating lymphocytes (TIL) present in the spheroids after treatment with IgG or the WM-A1-3389 antibody when IGSF1 overexpressing human lung cancer cell (NCI-H292 IGSF1 O/E) spheroids were co-cultured with human peripheral mononuclear cells (PBMC).

FIG. 10 is a graph showing the expression of HMGB1 and Hsp90 in the spheroids after treatment with IgG or the WM-A1-3389 antibody when IGSF1 overexpressing human lung cancer cells (NCI-H292 IGSF1 O/E) and the control (NCI-H292 MOCK) spheroids were co-cultured with human peripheral mononuclear cells (PBMC).

FIG. 11 is a graph showing a result obtained by measuring the tumor size of a group administered with IgG or the WM-A1-3389 antibody in a mouse model transplanted with IGSF1 overexpressing human lung cancer cells (NCI-H292 IGSF1 O/E).

FIG. 12 is a graph showing the tumor size of a mouse group administered with IgG or the WM-A1-3389 antibody by a subject in a mouse model transplanted with IGSF1 overexpressing human lung cancer cells (NCI-H292 IGSF1 O/E).

FIG. 13 illustrates a result obtained by analyzing the expression level of IGSF1 in Caucasian lung cancer patient tissue.

### BEST MODE FOR CARRYING OUT THE INVENTION

In one aspect of the present invention, there is provided an anticancer agent comprising an anti-IGSF1 antibody that specifically binds to the C-terminus of IGSF1 as an active ingredient.

As used herein, the term “IGSF1” is a membrane protein encoded by the IGSF1 gene found on the X chromosome of humans and other mammalian species. Although the function of IGSF1 in normal cells is not well known, IGSF1 mutation is known to cause diseases such as IGSF1 deficiency syndrome or central hypothyroidism.

In the present invention, the IGSF1 may be included without limitation as long as it is mammalian IGSF1, but preferably, it may refer to human IGSF1. In addition, in the present invention, the IGSF1 protein includes all of the native IGSF1 protein or variants thereof, but is not limited thereto. The native IGSF1 protein generally refers to a polypeptide comprising the amino acid sequence of the native IGSF1 protein, and the amino acid sequence of the native IGSF1 protein generally refers to an amino acid sequence found in a naturally occurring IGSF1. The information on the IGSF1 may be obtained from a known database such as GenBank of the National Institutes of Health of the United States of America, and for example, may have the amino acid sequence of Genebank accession number NP_001164433.1 or the amino acid sequence of SEQ ID NO: 19, but is not limited thereto.

As used herein, the term “anti-IGSF1 antibody” refers to an antibody capable of binding to IGSF1, and may be used interchangeably with an “antibody specific for IGSF1” in the present invention. In particular, the anti-IGSF1 antibody may specifically bind to the C-terminus of IGSF1. The form of the antibody may include both a whole antibody and an antibody fragment thereof.

As used herein, the term “anticancer agent” may include any composition or medicament that exhibits a preventive or therapeutic effect on cancer.

In the present invention, the anti-IGSF1 antibody that binds to the C-terminus of IGSF1 may effectively kill cancer in which IGSF1 is overexpressed. In this case, the cancer may be any one selected from the group consisting of gastric cancer, liver cancer, lung cancer, non-small cell lung cancer, colorectal cancer, bladder cancer, bone cancer, blood cancer, breast cancer, melanoma, thyroid cancer, parathyroid cancer, bone marrow cancer, rectal cancer, throat cancer, laryngeal cancer, esophageal cancer, pancreatic cancer, tongue cancer, skin cancer, brain tumor, uterine cancer, head or neck cancer, gallbladder cancer, oral cancer, perianal cancer, colon cancer, and central nervous system tumor, but is not limited thereto as long as it is a cancer in which IGSF1 is overexpressed.

### Anti-IGSF1 Antibody

In another aspect of the present invention, there is provided an antibody specific for IGSF1 or a fragment thereof, comprising a heavy chain variable region comprising H-CDR1 of SEQ ID NO: 1, H-CDR2 of SEQ ID NO: 2 and H-CDR3 of SEQ ID NO: 3; and a light chain variable region comprising L-CDR1 of SEQ ID NO: 4, L-CDR2 of SEQ ID NO: 5 and L-CDR3 of SEQ ID NO: 6.

As used herein, the term “antibody” refers to an immunoglobulin molecule that immunologically reacts with a specific antigen, and refers to a protein molecule that specifically recognizes an antigen. The antibody includes a whole antibody, a monoclonal antibody, a polyclonal antibody, a single domain antibody, a single chain antibody, a multispecific antibody, a human antibody, a humanized antibody, a chimeric antibody, an intrabody, scFv, an Fab fragment, an F (ab′) fragment, Fv (sdFv) linked by disulfide bonds and an epitope binding fragment of any of the above, but is not limited thereto.

The heavy and light chains of an immunoglobulin may include a constant region and a variable region, respectively.

The light and heavy chain variable regions of an immunoglobulin include three variable regions called complementarity determining regions (CDRs) and four framework regions (FRs). The CDRs mainly serve to bind to an epitope of an antigen. The CDRs of each chain are typically called CDR1, CDR2, and CDR3 sequentially, starting from the N terminus, and are identified by the chain in which a specific CDR is located. The antibody specific for IGSF1 and fragment thereof of the present invention may comprise a heavy chain variable region (VH) comprising H-CDR1 of SEQ ID NO: 1, H-CDR2 of SEQ ID NO: 2 and H-CDR3 of SEQ ID NO: 3. In addition, the antibody specific for IGSF1 and fragment thereof of the present invention may comprise a light chain variable region (VL) comprising L-CDR1 of SEQ ID NO: 4, L-CDR2 of SEQ ID NO: 5 and L-CDR3 of SEQ ID NO: 6. In this case, the heavy chain variable region may have the amino acid sequence of SEQ ID NO: 7, and the light chain variable region may have the amino acid sequence of SEQ ID NO: 8. The antibody herein may be referred to as WM-A1-3389.

The heavy chain variable region of the antibody may comprise or consist of an amino acid sequence having about 90%, about 91%, about 92%, about 93%, about 94%, about 95%, about 96%, about 97%, about 98%, about 99%, or about 100% identity to the amino acid sequence of SEQ ID NO: 7. In addition, the light chain variable region of the antibody may comprise or consist of an amino acid sequence having about 90%, about 91%, about 92%, about 93%, about 94%, about 95%, about 96%, about 97%, about 98%, about 99% or about 100% identity to the amino acid sequence of SEQ ID NO: 8.

The heavy chain constant region (CH) of an immunoglobulin exhibits different amino acid compositions and sequences and thus possess different types of antigenicity. Therefore, immunoglobulins may be classified into five categories and may be referred to as immunoglobulin isotypes, i.e., IgM, IgD, IgG, IgA and IgE. The corresponding heavy chains are the μ chain, the δ chain, the γ chain, the a chain and the ε chain, respectively. In addition, depending on the amino acid composition of the hinge region and the number and position of heavy chain disulfide bonds, the same type of Ig may be classified into different subtypes. For example, IgG may be classified into IgG1, IgG2, IgG3 and IgG4. Light chains may be classified into a κ or λ chain according to different constant regions. Each of the five types of IgG may have either a κ or a λ chain.

When the antibody specific for IGSF1 of the present invention includes a constant region, it may include a constant region that is derived from IgG, IgA, IgD, IgE, IgM, or a partial hybrid thereof.

As used herein, the term “hybrid” refers to the presence of sequences corresponding to immunoglobulin heavy chain constant regions having two or more different origins within a single chain immunoglobulin heavy chain constant region. For example, hybridization of domains consisting of 1 to 4 domains selected from the group consisting of CH1, CH2 and CH3 of IgG, IgA, IgD, IgE and IgM is possible.

In addition, when the antibody specific for IGSF1 of the present invention comprises a light chain constant region (LC), the light chain constant region may be derived from a λ or κ light chain.

As used herein, the term “fragment of antibody” refers to an Fab fragment, an Fab′ fragment, an F(ab′)₂ fragment having antigen-binding activity, as well as an scFv fragment which is Fv fragment that binds to IGSF1, and includes CDR regions of the antibodies described in the present invention. The Fv fragment is the smallest fragment of antibody, comprising a heavy chain variable region and a light chain variable region, without constant regions, and possessing all antigen-binding sites.

### Polynucleotide Encoding Anti-IGSF1 Antibody

In another aspect of the present invention, there is provided a polynucleotide encoding an antibody specific for IGSF1 or a fragment thereof. The anti-IGSF1 antibody and fragment thereof are as described above. In this case, the heavy chain region of the polynucleotide may comprise the nucleotide sequence of SEQ ID NO: 9, and the light chain region may comprise the nucleotide sequence of SEQ ID NO: 10.

If the polynucleotide encodes the same polypeptide, one or more bases may be mutated by substitution, deletion, insertion or a combination thereof. When the polynucleotide sequence is prepared by chemical synthesis, synthesis methods well known in the art, for example, the method described in the literature (Engels and Uhlmann, Angew Chem IntEd Engl., 37:73-127, 1988) may be used, and may include triester, phosphite, phosphoramidite and H-phosphate methods, PCR and other auto-primer methods, synthesis methods of oligonucleotide on a solid support, and the like.

According to one embodiment, the polynucleotide may comprise a nucleotide sequence having at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% identity to the nucleotide sequence of SEQ ID NO: 9 or SEQ ID NO: 10, respectively.

The polynucleotide may further comprise a signal sequence or a leader sequence. As used herein, the term “signal sequence” refers to a nucleic acid encoding a signal peptide that directs secretion of a target protein. The signal peptide is translated and then cleaved in a host cell. Specifically, the signal sequence of the present invention is nucleotides encoding an amino acid sequence that initiates the transports of a protein across the ER (endoplasmic reticulum) membrane.

Signal sequences are well known in the art for their characteristics. Such signal sequences typically contain 16 to 30 amino acid residues, and may contain more or fewer amino acid residues than such amino acid residues. A conventional signal peptide is composed of three regions, that is, a basic N-terminal region, a central hydrophobic region, and a more polar C-terminal region. The central hydrophobic region contains 4 to 12 hydrophobic residues that cause the signal sequence to be immobilized during transports of an immature polypeptide through the membrane lipid bilayer.

After initiation, the signal sequence is cleaved in the lumen of the ER by intracellular enzymes, commonly known as signal peptidases. In this case, the signal sequence may be a secretory signal sequence of tPa (tissue plasminogen activation), HSV gDs (signal sequence of herpes simplex virus glycoprotein D), an IgG signal sequence or a growth hormone. Preferably, a secretory signal sequence used in higher eukaryotic cells including mammals and the like may be used.

The signal sequence useful in the present invention includes antibody light chain signal sequences, such as antibody 14.18 (Gillies et al., J. Immunol. Methods, 1989. 125:191-202), antibody heavy chain signal sequences, such as MOPC141 antibody heavy chain signal sequence (Sakano et al., Nature, 1980. 286: 676-683) and other signal sequences known in the art (see, e.g., Watson et al., Nucleic Acid Research, 1984. 12:5145-5164).

Vector Loaded with Polynucleotide

In another aspect of the present invention, there is provided a vector comprising a polynucleotide encoding the antibody specific for IGSF1 or fragment thereof. The heavy chain region of the polynucleotide may comprise the nucleotide sequence of SEQ ID NO: 9, and the light chain region may comprise the nucleotide sequence of SEQ ID NO: 10. In addition, the polynucleotide may further comprise a signal sequence or a leader sequence. Herein, an antibody specific for IGSF1 and a fragment thereof, and a signal sequence are as described above.

In this case, the vector may be two vectors containing the polynucleotides of the heavy chain and the light chain, respectively, or a bicistronic vector containing both the polynucleotides.

As used herein, the term “vector” may be introduced into a host cell to be recombined with and inserted into the genome of the host cell. Alternatively, the vector is understood as nucleic acid means containing a nucleotide sequence which is spontaneously replicable as an episome. The vectors include linear nucleic acids, plasmids, phagemids, cosmids, RNA vectors, viral vectors, mini-chromosomes, and analogs thereof. Examples of the viral vector include retroviruses, adenoviruses, and adeno-associated viruses, but are not limited thereto.

Specifically, the vector may include plasmid DNA, phage DNA, and the like; and commercially developed plasmids (pUC18, pBAD, pIDTSAMRT-AMP, and the like), E. coli-derived plasmids (pYG601BR322, pBR325, pUC118, pUC119, and the like), Bacillus subtilis-derived plasmids (pUB110, pTP5, and the like), yeast-derived plasmids (YEp13, YEp24, YCp50, and the like), phage DNA (Charon4A, Charon21A, EMBL3, EMBL4, λgt10, λgt11, λZAP, and the like), animal viral vectors (retroviruses, adenoviruses, vaccinia viruses, and the like), insect viral vectors (baculoviruses and the like) and the like. Since the vector exhibits different expression levels and modification and the like of a protein depending on a host cell, it is preferred to select and use a host cell which is most suitable for the purpose.

In addition, the plasmid may contain a selectable marker such as an antibiotic resistance gene, and host cells retaining the plasmid may be cultured under selective conditions.

As used herein, the term “gene expression” or “expression” of a target protein is understood to mean transcription of DNA sequences, translation of mRNA transcripts, and secretion of fusion protein products or fragments thereof. A useful expression vector may be RcCMV (Invitrogen, Carlsbad) or a variant thereof. The expression vector may contain a human cytomegalovirus (CMV) promoter for promoting continuous transcription of a target gene in mammalian cells, and a bovine growth hormone polyadenylation signal sequence for increasing the stability level of RNA after transcription.

### Transformed Cell Expressing Anti-IGSF1 Antibody

In another aspect of the present invention, there is provided a transformed cell into which an expression vector comprising a polynucleotide encoding the antibody specific for IGSF1 or fragment thereof is introduced. The antibody specific for IGSF1 and fragment thereof are as described above.

As used herein, the term “transformed cell” refers to a prokaryotic cell and a eukaryotic cell into which a recombinant expression vector may be introduced. The transformed cell may be constructed by introducing a vector into a host cell and transforming it. In addition, the fusion protein of the present invention may be produced by expressing the polynucleotide included in the vector.

The transformation may be performed by various methods. As long as it may produce the fusion protein of the present invention, it is not particularly limited thereto. Specifically, the transformation method, for example, CaCl₂) precipitation method, Hanahan method whose efficiency has been increased by using a reducing agent such as dimethyl sulfoxide (DMSO) in CaCl₂) precipitation method, electroporation, calcium phosphate precipitation method, protoplast fusion method, agitation method using silicon carbide fiber, agrobacterium mediated transformation method, transformation method using PEG, dextran sulfate, lipofectamine and dry/inhibition mediated transformation method, and the like may be used. In addition, by using the infection as a means, a target object may be delivered into a cell using virus particles. In addition, a vector may be introduced into a host cell by gene bombardment or the like.

In addition, as long as the host cell used for the construction of the transformed cell may also produce the fusion protein of the present invention, it is not particularly limited thereto. Specifically, the host cell may include, but is not limited to, prokaryotic cells, eukaryotic cells, and cells of mammalian, plant, insect, fungal, or bacterial origin. As an example of the prokaryotic cells, E. coli may be used. In addition, as an example of the eukaryotic cells, yeast may be used. In addition, for the mammalian cells, CHO cells, F2N cells, COS cells, BHK cells, Bowes melanoma cells, HeLa cells, 911 cells, AT1080 cells, A549 cells, SP2/0 cells, human lymphoblastoids, NSO cells, HT-1080 cells, PERC.6 cells, HEK293 cells, HEK293T cells or the like may be used, but are not limited thereto. Any cells which are known to those of ordinary skill in the art to be usable as mammalian host cells may be used.

As described above, for optimization of properties of an anti-IGSF1 antibody and a fragment thereof as a therapeutic agent or for any other purpose, glycosylation pattern (for example, sialic acid, fucosylation, glycosylation) of the fusion protein may be adjusted by manipulating glycosylation-related genes possessed by host cells through methods known to those of ordinary skill in the art.

### Method of Producing Anti-IGSF1 Antibody

In another aspect of the present invention, there is provided a method of producing the antibody specific for IGSF1 or fragment thereof. In this case, the antibody specific for IGSF1 and fragment thereof are as described above.

A method of producing the fusion protein may comprise i) culturing the transformed cell; and ii) recovering the anti-IGSF1 antibody or fragment thereof of the present invention.

As used herein, the term “culture” refers to a method of growing microorganisms in an appropriately artificially controlled environmental condition.

A method of culturing the transformed cell may be carried out using methods well known in the art. Specifically, the culture is not particularly limited as long as it may be produced by expressing the fusion protein of the present invention. Specifically, the culture may be carried out in a batch process, or carried out continuously in a fed batch or repeated fed batch process.

In addition, recovering the fusion protein dimer from the culture may be performed by methods known in the art. Specifically, the recovery method is not particularly limited as long as it may recover the produced fusion protein of the present invention. Preferably, the recovery method may be methods such as centrifugation, filtration, extraction, spraying, drying, evaporation, precipitation, crystallization, electrophoresis, fractional dissolution (for example, ammonium sulfate precipitation), chromatography (for example, ion exchange, affinity, hydrophobicity, and size exclusion) and the like.

### Use of Anti-IGSF1 Antibody

In another aspect of the present invention, there is provided a pharmaceutical composition for preventing or treating cancer, comprising the IGSF1 antibody or fragment thereof as an active ingredient.

In this case, the cancer may be a cancer in which IGSF1 is overexpressed. In addition, the cancer may be any one selected from the group consisting of gastric cancer, liver cancer, lung cancer, non-small cell lung cancer, colorectal cancer, bladder cancer, bone cancer, blood cancer, breast cancer, melanoma, thyroid cancer, parathyroid cancer, bone marrow cancer, rectal cancer, throat cancer, laryngeal cancer, esophageal cancer, pancreatic cancer, tongue cancer, skin cancer, brain tumor, uterine cancer, head or neck cancer, gallbladder cancer, oral cancer, perianal cancer, colon cancer, and central nervous system tumor.

The term “prevention” refers to any action that inhibits the occurrence of cancer or delays the onset of cancer by administration of the pharmaceutical composition. The term “treatment” refers to any action that improves or beneficially changes the symptoms of cancer by administration of the pharmaceutical composition.

In the pharmaceutical composition for the prevention or treatment of cancer of the present invention, the anti-IGSF1 antibody or fragment thereof may be included in any amount (effective amount) depending on the use, formulation, purpose of combining, and the like, as long as it may exhibit an anticancer activity. Herein, “effective amount” refers to an amount of an active ingredient capable of inducing an anticancer effect. Such an effective amount may be determined experimentally within the ordinary ability of those of ordinary skill in the art. The pharmaceutical composition of the present invention may comprise the antibody as an active ingredient in an amount of from about 0.1% by weight to about 90% by weight, specifically from about 0.5% by weight to about 75% by weight, more specifically from about 1% by weight to about 50% by weight based on the total weight of the composition.

The pharmaceutical composition of the present invention may comprise a conventional, non-toxic pharmaceutically acceptable carrier to be combined into a formulation according to a conventional method.

The pharmaceutically acceptable carrier may be any non-toxic material suitable for delivery to a patient. Distilled water, alcohol, fats, waxes and inert solids may be included as a carrier. In addition, a pharmaceutically acceptable adjuvant (buffering agent, dispersing agent) may be included in the pharmaceutical composition.

As used herein, the term “pharmaceutically acceptable carrier” refers to a carrier or diluent that does not irritate the organism and does not inhibit the biological activity and property of the administered compound. Pharmaceutically acceptable carriers for compositions formulated as liquid solutions are sterile and biocompatible, and saline, sterile water, Ringer's solution, buffered saline, albumin injection solution, dextrose solution, maltodextrin solution, glycerol, ethanol, and a mixture of one or more components of these components may be used, and other conventional additives such as sweeteners, solubilizers, wetting agents, emulsifiers, isotonic agents, absorbents, antioxidants, preservatives, lubricants, fillers, buffers, and bacteriostats may be added as needed.

The composition of the present invention may be prepared in a variety of formulations for parenteral administration (such as, intramuscular, intravenous or subcutaneous injection). When the pharmaceutical composition of the present invention is prepared as a parenteral formulation, it may be formulated in the form of injections, transdermal preparations, nasal inhalants and suppositories together with a suitable carrier according to methods known in the art. Preparations for injection include sterile aqueous solutions, non-aqueous solutions, suspensions, emulsions, freeze-dried preparations and suppositories. Non-aqueous solvents and suspending agents include propylene glycol, polyethylene glycol, vegetable oils such as olive oil, and injectable esters such as ethyl oleate. As the base of the suppositories, Witepsol, Macrogol, Tween 61, cacao butter, laurin, glycerogelatin, and the like may be used. On the other hand, injections may include conventional additives such as solubilizers, isotonic agents, suspending agents, emulsifiers, stabilizers, and preservatives.

Formulation of a pharmaceutical composition is known in the art, and specifically, reference may be made to the literature [Remington's Pharmaceutical Sciences (19th ed., 1995)] and the like. The literature is considered a part of the present specification.

The antibody or composition of the present invention may be administered to a patient in a therapeutically effective amount or in a pharmaceutically effective amount.

As used herein, the term “administration” means introducing a predetermined substance to a subject by an appropriate method, and the composition may be administered through any general route as long as it may reach a target tissue. The route of administration may include intraperitoneal administration, intravenous administration, intramuscular administration, subcutaneous administration, intradermal administration, topical administration, intranasal administration, and intrarectal administration, but is not limited thereto.

Herein, “therapeutically effective amount” or “pharmaceutically effective amount” refers to an amount of an antibody or composition effective for preventing or treating a target disease, and means an amount that is sufficient to treat a disease with a reasonable benefit/risk ratio applicable to medical treatment and does not cause side effects. The level of the effective amount may be determined according to the patient's health condition, the type and severity of the disease, the activity of the drug, the sensitivity to the drug, administration method, administration time, the route of administration and excretion rate, treatment duration, factors including the combined or concurrently used drugs, and other factors well known in the medical field.

Specifically, the effective amount of the antibody in the composition of the present invention may vary depending on the age, sex, and body weight of the patient, and in general, may be administered from about 0.1 mg to about 1,000 mg, or from about 5 mg to about 200 mg per kg of body weight daily or every other day or may be divided into 1 to 3 times a day. However, since it may be increased or decreased depending on the route of administration, the severity of the disease, sex, body weight, age, and the like, the scope of the present invention is not limited thereto.

The term “subject” refers to a subject to which the composition of the present invention may be applied (prescribed), and may be a mammal, such as a rat, a mouse, or a livestock, including a human. Preferably, it may be a human, but is not limited thereto.

The antibody of the present invention or a pharmaceutical composition comprising the same may be administered as an individual therapeutic agent or in combination with other therapeutic agents, may be administered sequentially or simultaneously with conventional therapeutic agents, or may be administered singly or multiple times. In this case, the other therapeutic agents may further include any compound or natural extract known to have anticancer activity and safety has already been verified for the enhancement and reinforcement of anticancer activity.

Taking all of the above factors into consideration, it is important to administer an amount that may obtain the maximum effect with the minimum amount of side effects or without side effects, which may be easily determined by those of ordinary skill in the art.

In another aspect of the present invention, there is provided the use of an antibody specific for IGSF1 or a fragment thereof for the manufacture of a medicament for preventing or treating cancer, wherein the anti-IGSF1 antibody and fragment thereof, cancer, prevention and treatment are the same as described above.

In another aspect of the present invention, there is provided the use of an antibody specific for IGSF1 or a fragment thereof for the prevention and treatment of cancer, wherein the anti-IGSF1 antibody and fragment thereof, cancer, prevention and treatment are the same as described above.

In another aspect of the present invention, there is provided a method for preventing and treating cancer, comprising administering to a subject an antibody specific for IGSF1 or a fragment thereof, wherein the anti-IGSF1 antibody and fragment thereof, cancer, administration, treatment and prevention are the same as described above.

The subject may be a mammal, preferably a human. In addition, the subject may be a cancer patient or a subject who is highly likely to suffer from cancer.

The route of administration, dosage, and frequency of administration of the antibody specific for IGSF1 or fragment thereof may vary depending on the patient's condition and the presence or absence of side effects, and thus the antibody specific for IGSF1 or fragment thereof may be administered to a subject in various ways and amounts. The optimal administration method, dosage, and frequency of administration may be selected in an appropriate range by those of ordinary skill in the art. In addition, the antibody specific for IGSF1 or fragment thereof may be administered in combination with other drugs or physiologically active substances whose therapeutic effect is known with respect to cancer, or may be formulated in the form of combination preparations with other drugs.

### MODE FOR CARRYING OUT THE INVENTION

Hereinafter, the present invention will be described in more detail by way of the following examples. However, the following examples are only for illustrating the present invention, and the scope of the present invention is not limited to the following examples.

### Example 1. Construction of Anti-IGSF1 Antibody

### Example 1.1. Expression and Purification of IGSF1 Antigen

Only the extracellular domain of IGSF1 was amplified in the Jurkat cell cDNA library through PCR method, and then the human Fc (fragment crystallizable region) and His-tag were fused at the carboxy terminus (C-terminus) using the N293F vector (YBiologics Co., Ltd.) to construct an IGSF1 protein expression vector. The HEK293F cells were transfected with the constructed IGSF1 expression vector and then cultured for 6 days in a medium to which 1 mM valporic acid (valproate) was added. Then, the IGSF1 extracellular domain was subjected to primary purification using protein A agarose, and then the IGSF1 extracellular domain was subjected to secondary purification using Superdex 200 gel filtration chromatography, and then used for antibody screening.

### Example 1.2. Screening of IGSF1 Human Antibody

After coating and blocking of the IGSF1 antigen, bio-panning (YBiologics Co., Ltd.) was performed using the prepared human antibody library phages (YBiologics Co., Ltd.) to elute only the phages that were specifically bound to the antigen. The second and third rounds of bio-panning were performed with the phage amplified in the first round of bio-panning. ELISA was performed to confirm the specificity with the antigen for the positive phage antibody pool obtained through each round of bio-panning. In addition, it was confirmed that the anti-IGSF1 antibody was enriched in the phage pool obtained through the third round. Hundred types of monoclones with high binding capacity were selected from the third round of panning in each polyphage ELISA, and they were used to confirm whether they specifically bind to IGSF1 through ELISA analysis, thereby obtaining preliminary antibody clones. The screending preliminary antibody clones were subjected to DNA nucleotide sequencing to select 99 types of phages having different nucleotide sequences. It was confirmed that the selected 99 positive phage clones strongly bound to the antigen IGSF1, but did not bind to other antigens. Through the above method, as a result of screening antibodies that show specificity for the IGSF1 antigen using various other antigens, a total of 95 types could be selected.

### Example 1.3. Confirmation of Specificity for IGSF1 Antigen

The selected antibodies were compared and analyzed for the specificity for other antigens including IGSF1 by ELISA method. It was confirmed that whether phage clones bind to various types of unspecified antigens such as mFc, hRAGE-Fc, CD58-Fc, and ITGA6-Fc, which were control antigens. The antibodies thus obtained were converted from phage to an IgG whole vector, and it was confirmed that the heavy chain sequence and the light chain sequence of the converted 95 clones matched the sequence of the phage antibody. Among the obtained antibodies, the most optimized antibody was selected, and it was referred to as “WM-A1-3389.” The CDR sequences of the WM-A1-3389 antibody are shown in Table 1 below.

TABLE 1
WM-A1-3389

| CDR | Amino acid sequence | SEQ ID NO |
|---|---|---|
| H-CDR1 | GGTFSTYA | 1 |
| H-CDR2 | IIPFVGTV | 2 |
| H-CDR3 | VRDGGRSYFDS | 3 |
| L-CDR1 | TSNIGSNL | 4 |
| L-CDR2 | DNH | 5 |
| L-CDR3 | VAWDDSLNGYV | 6 |

### Example 1.4. Production of WM-A1-3389 Antibody

In order to produce the WM-A1-3389 antibody, a polynucleotide (SEQ ID NO: 23) encoding a heavy chain (SEQ ID NO: 21) was loaded into the N293F vector (YBiologics Co., Ltd.) (hereinafter referred to as ‘HC DNA’). In addition, a polynucleotide (SEQ ID NO: 24) encoding a light chain (SEQ ID NO: 22) was loaded into the N293F vector (YBiologics Co., Ltd.) (hereinafter referred to as LC DNA). The vector was transformed into the cells, and then the WM-A1-3389 antibody was obtained and purified. The purified protein was identified by SDS-PAGE.

TABLE 2

| | Amino acid sequence | SEQ ID NO |
|---|---|---|
| Heavy chain | QVQLVQSGAEVKRPGSSVKVSCKASGGTFSTYAISWV | 21 |
| | RQAPGQGLEWMGRIIPFVGTVDYAQKFQDRVTITADK | |
| | STNTAYMELSSLRSEDTAVYYCVRDGGRSYFDSWGPG | |
| | ILVTVSSASTKGPSVFPLAPSSKSTSGGTAALGCLVKDY | |
| | FPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVV | |
| | DVSHEDPEVKFNWYVDGVEVHNAKTKPREEQYNSTY | |
| | RVVSVLTVLHQDWLNGKEYKCKVSNKALPAPIEKTIS | |
| | KAKGQPREPQVYTLPPSREEMTKNQVSLTCLVKGFYPS | |
| | DIAVEWESNGQPENNYKTTPPVLDSDGSFFLYSKLTVD | |
| | KSRWQQGNVFSCSVMHEALHNHYTQKSLSLSPGK | |
| Light chain | QFVLTQPPSVSAAPGQDVIISCSGNTSNIGSNLVSWFQQ | 22 |
| | FPETAPKLLIYDNHKRPSGISDRFSGTKSGTSASLAISGL | |
| | QSEDEADYYCVAWDDSLNGYVFGTGTKVTVLRTVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |

### Example 2. Analysis of Relationship Between IGSF1 Expression and Tumor-Infiltrating Lymphocytes

### Example 2.1. Construction of IGSF1 Overexpressing Cell Line

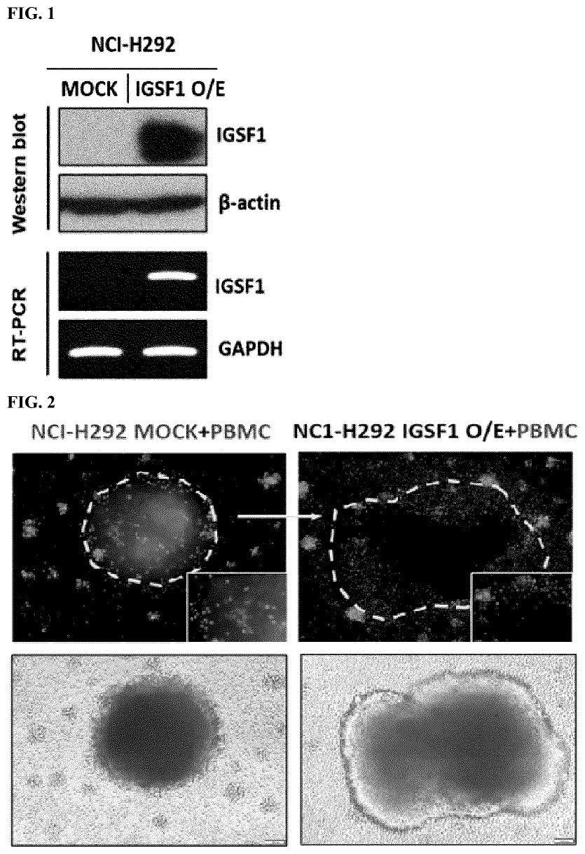

IGSF1 was overexpressed in the human lung cancer cell line NCI-H292 or the human embryonic kidney cell line HEK293E cells to construct a cell line in which IGSF1 is overexpressed (FIG. 1). In this case, MOCK is a control without IGSF1 expression.

Specifically, an expression vector (OriGene Technologies, Inc., Cat No. RC209621) comprising a polynucleotide encoding IGSF1 was transfected into the human lung cancer cell line NCI-H292 cells or the human embryonic kidney cell line HEK293F cells. Thereafter, the transfected cells were selected by culturing them in a medium containing G418 (neomycin). The expression level of IGSF1 was checked for the selected clones, and the clone showing the highest IGSF1 expression was selected and used for the experiment. MOCK refers to an empty vector into which a polynucleotide encoding IGSF1 is not loaded.

### Example 2.2. Analysis of Relationship Between IGSF1 Expression and Tumor-Infiltrating Lymphocytes in Lung Cancer Cell Line Spheroids

In order to confirm the correlation between IGSF1 expression and tumor-infiltrating lymphocytes (TIL) in lung cancer at a cellular level, tumor-infiltrating lymphocytes were identified in spheroids using lung cancer cells in which IGSF1 was overexpressed.

First, in order to construct lung cancer cell spheroids, NCI-H292 IGSF1 O/E cells and NCI-H292 MOCK cells were seeded in a U-Bottom 96-well plate (Nunc, 174925) to 2×10⁴ cells/well, respectively, and cultured for 72 hours in a carbon dioxide incubator at 37° C. Peripheral blood mononuclear cells (PBMC) were prepared by removing the supernatant by centrifugation at 1,200 rpm for 10 minutes and resuspending in PBS. 18 μl of DMSO was added to CFSE (carboxyfluorescein succinimidyl ester, Invitrogen, C34554) to a concentration of 5 mM and diluted in PBS to 1 mM. 1 μl of 1 mM CFSE solution was added per 1×10⁶ cells/ml of the prepared peripheral blood mononuclear cells and then stained in a carbon dioxide incubator at 37° C. for 10 minutes. Then, a medium containing FBS (fetal bovine serum) in an amount of 5 times the amount of PBS was added to a solution containing the stained peripheral blood mononuclear cells (PBMC). Thereafter, the reaction was carried out in a carbon dioxide incubator at 37° C. for 5 minutes. The supernatant was removed by centrifugation at 1,200 rpm for 10 minutes, and then the stained peripheral blood mononuclear cells were resuspended in a medium to prepare peripheral blood mononuclear cells for use in the experiment.

Thereafter, the formed spheroids were transferred to an ultra-low adhesion 96-well plate (Corning, CLS3474) by 2 wells, and then seeded with peripheral blood mononuclear cells, which were stained with CFSE, to 1×10⁵ cells/well, and co-cultured in a carbon dioxide incubator at 37° C. for 24 hours. Tumor-infiltrating lymphocytes (TIL) were observed under a fluorescence microscope. In this case, the NCI-H292 MOCK cells were used as a control for IGSF1 overexpressing cells.

As a result, it was confirmed that tumor-infiltrating lymphocytes (TIL) were decreased in IGSF1 overexpressing human lung cancer cell (NCI-H292 IGSF1 O/E) spheroids compared to the control (NCI-H292 MOCK) (FIG. 2).

### Example 2.3. Analysis of Relationship Between IGSF1 Expression and Tumor-Infiltrating Lymphocytes in Tumor Tissues of Humanized Mice Transplanted with Lung Cancer Cell Lines

In order to confirm the correlation between the expression of IGSF1 and tumor-infiltrating lymphocytes (TIL) in lung cancer at an in vivo level, tumor-infiltrating lymphocytes were identified in the tumor tissues of humanized mice transplanted with human lung cancer cells in which IGSF1 was overexpressed.

Specifically, NCI-H292 IGSF1 O/E cells, an IGSF1 overexpressing human lung cancer cell line, or NCI-H292 MOCK cells, a control, were transplanted at 5×10⁶ cells per animal into NSG mice (SID (NSGA) mice, F) transplanted with human peripheral blood mononuclear cells (PBMC), and then tumor sections from the mice were collected on day 17.

Human peripheral blood mononuclear cells were isolated from each collected tumor, analyzed by FACS, and tumor-infiltrating lymphocytes and the expression level of IGSF1 in the tumor tissue were confirmed by immunohistochemistry staining of the tumor tissue. In order to identify the human peripheral blood mononuclear cells infiltrating the tumor, first, the tumor tissue was treated with collagenase B (Roche, cat. #11088815001) and reacted at 37° C. for 2 hours or more to dissociate the tumor tissue. When the tumor tissue was completely dissociated into cells, it was isolated into single cells by pipetting with a 1 ml pipette.

The isolated single cells were transferred to a 50 ml tube (SPL, cat. #50050) and then washed with 20 ml of PBS. Thereafter, the supernatant was removed by centrifugation at 1,200 rpm for 3 minutes. The remaining cells were reacted with DNase I (Roche, cat. #11284932001) at 37° C. for 20 minutes. Thereafter, 20 ml of PBS was added, and the supernatant was removed by centrifugation at 1,200 rpm for 3 minutes. The remaining cells were treated with 0.25% Trysin/EDTA (GIBCO, cat. #15400-054). The cells were mixed well, and then a cell strainer (SPL, cat. #93070) was placed on a new 50 ml tube, and the cells were filtered. 20 ml of PBS was added to the tube containing the filtered cells and mixed well. Thereafter, the supernatant was removed by centrifugation at 1,200 rpm for 3 minutes, and then 1 ml of Stain Buffer (BD, cat. #554656) was added to the remaining cells and washed.

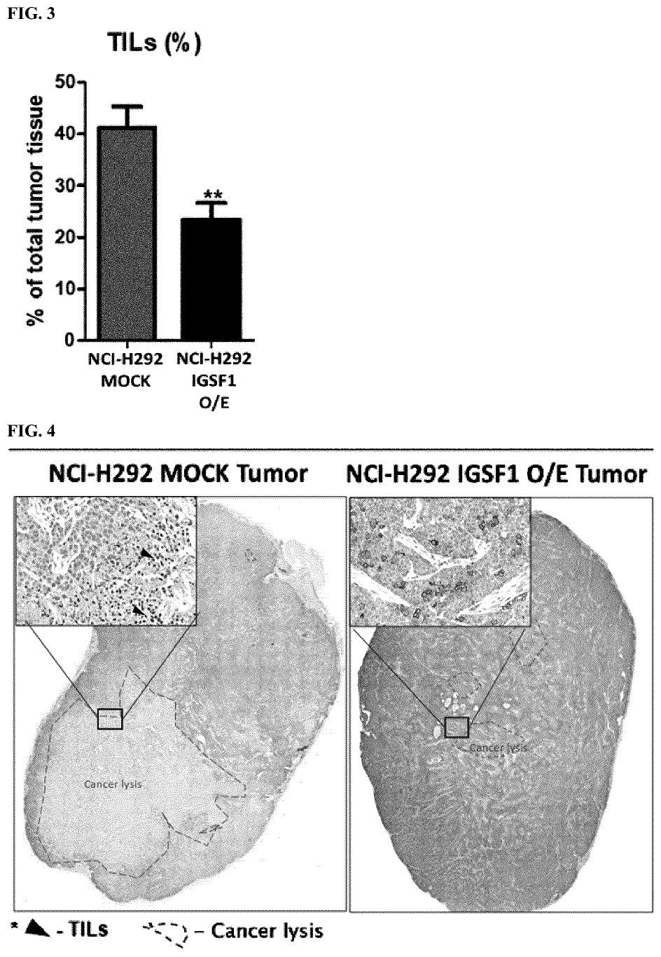

In order to block the non-specific antibody reaction of the isolated cells, 2 μg of human Fc block (BD, cat. #564219) was added and reacted for 10 minutes at room temperature. After the reaction, an anti-human CD45 (BD, cat. #564357) antibody was added and reacted at 4° C. for 30 minutes while blocking the light. After the reaction, 1 ml of Stain Buffer was added and washed. Thereafter, the supernatant was removed by centrifugation at 1,200 rpm for 3 minutes, and the cells were collected, and then 200 μl of Stain Buffer was added and analyzed by a BD LSRFortessa™ flow cytometer (FIG. 3).

In addition, the distribution of tumor lymphocytes expressed in the tumor and the expression of IGSF1 were confirmed by immunohistochemistry staining method. Specifically, NCI-H292 IGSF1 O/E cells, a human lung cancer cell line, or NCI-H292 MOCK cells were transplanted at 5×10⁶ cells per animal into NSG mice (SID (NSGA) mice, F) transplanted with human peripheral blood mononuclear cells (PBMC). Thereafter, on day 17, sections of the tumor tissue collected from mice were deparaffinized and rehydrated.

Thereafter, it was soaked in a target repair buffer and heated in a microwave for 15 minutes for heat-induced epitope repair. Then, it was placed in a target repair buffer for 30 minutes, and then washed 3 times with Tris buffered saline-0.05% Tween 20 (TBS-T), and blocked with a blocking solution for 60 minutes. The primary antibody was an anti-IGSF1 antibody (Santacruz, sc-393786), which was 1:100 diluted and allowed to be bound overnight at 4° C. The next day, it was washed 3 times with TBS-T, and reacted with an endogenous peroxidase blocking reagent (Cell Marque, 925B) at room temperature for 5 minutes, and then the secondary antibody (Vector, PK-6101 PK-6102) was allowed to be bound at room temperature for 60 minutes. It was washed 3 times with TBS-T and then reacted with avidin-biotin for 60 minutes. The final DAB staining (Vector, SK-4100) was performed, and then the tissue staining was finished through a dehydration process, and the stained tissue sections were observed under a microscope.

As a result, it was confirmed that hCD45+ cells, which are human immune cells, were reduced in the tumor tissue of humanized mice transplanted with IGSF1 overexpressing human lung cancer cells (NCI-H292 IGSF1 O/E) compared to the control (NCI-H292 MOCK) (FIG. 4).

### Example 3. Analysis of Binding Affinity of Anti-IGSF1 Antibody

### Example 3.1. Analysis of Binding Affinity of Anti-IGSF1 Antibody at In Vitro

The binding affinity of the WM-A1-3389 antibody to the IGSF1 antigen was confirmed at an in vitro using ELISA analysis.

Specifically, it was treated with 100 ng of IGSF1 to coat a 96-well plate, and then 200 μl of 4% skim milk (PBST) was added and blocked at room temperature for 1 hour. The WM-A1-3389 antibody was serially diluted to 12 concentrations by ⅓ in 4% skim milk (PBST), treated, and then reacted at room temperature for 2 hours. After the reaction was completed, the wells were washed with PBST, treated with human IgG Fc-HRP antibody, and reacted at room temperature for 1 hour. Then, the wells were washed with PBST, and then TMB peroxidase substrate was added to confirm the degree of color development, and then absorbance was measured at 450 nm, and the results were compared and analyzed.

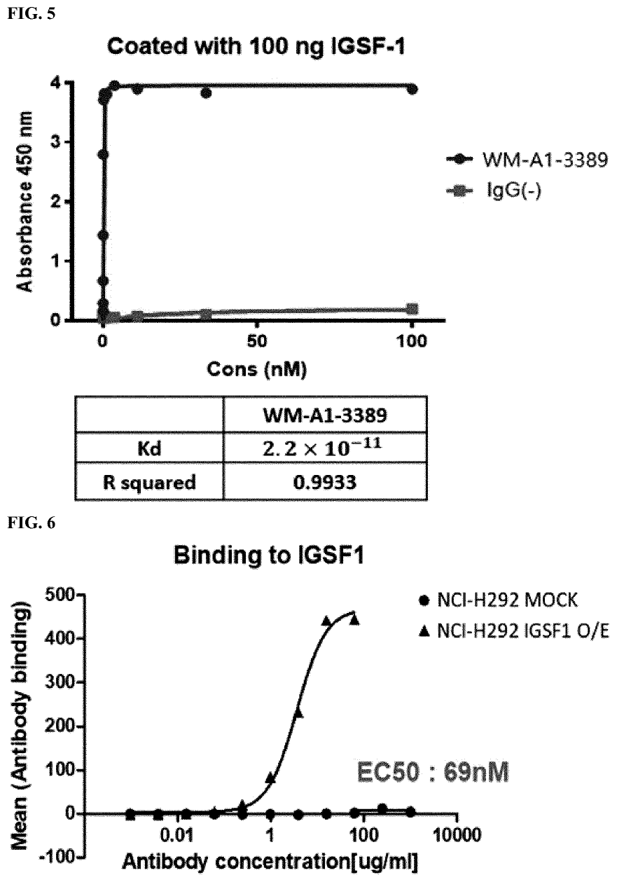

As a result, the WM-A1-3389 antibody had a Kd value of about 2.2× 10-11, indicating that it had a high binding affinity to the IGSF1 antigen (FIG. 5).

### Example 3.2. Analysis of Binding Affinity of Anti-IGSF1 Antibody to IGSF1 in Cells

In order to confirm the binding affinity of the WM-A1-3389 antibody to the IGSF1 antigen at a cellular level, the binding capacity of the WM-A1-3389 antibody to IGSF1 was confirmed using IGSF1 overexpressing human lung cancer cells (NCI-H292 IGSF1 O/E) and the control (NCI-H292 MOCK).

Specifically, the medium of NCI-H292 IGSF1 O/E cells and NCI-H292 MOCK cells was removed, washed once with PBS, and then treated with 2 ml of 0.25% trypsin-EDTA, and the cells were isolated. The isolated cells were diluted with 8 ml of PBS (hereinafter referred to as FACS buffer) containing 2% FBS and 0.05% sodium azide, and then the supernatant was removed by centrifugation at 1,200 rpm for 1 minute. Then, the cells were resuspended in FACS buffer to 1×10⁵ cells/ml. Thereafter, 1 ml of each was dispensed in a FACS tube, and the supernatant was removed by centrifugation at 1,200 rpm for 1 minute.

The pellet remaining in the FACS tube was released by vortexing, and the WM-A1-3389 antibody was diluted by ¼ from 20 μM to 0 μM per 200 μl of FACS buffer, added to a total of 12 concentrations, and then reacted at 4° C. for 30 minutes. After the reaction was completed, 1 ml of FACS buffer was added to each tube, and the supernatant was removed by centrifugation at 1,200 rpm for 1 minute. This process was performed a total of two times. The pellet remaining in the FACS tube was released by vortexing, and 5 μg/ml of FITC-labeled goat anti-human IgG antibody (Invitrogen, 62-8411) was added per 200 μl of FACS buffer and reacted while blocking the light at 4° C. for 30 minutes. After the reaction was completed, 1 ml of FACS buffer was added to each tube, and the supernatant was removed by centrifugation at 1,200 rpm for 1 minute. This process was performed a total of two times.

Finally, after removal of the supernatant, the remaining pellet was resuspended in 200 μl of FACS buffer and analyzed by FACS. For FACS analysis, FITC fluorescence value labeled in each cell was measured using a BD LSRFortessa™ flow cytometer, and then the results were analyzed using a FlowJo software, and EC50 value was calculated using a sigma plot program. In this case, NCI-H292 MOCK cells were used as a control for IGSF1 overexpressing cells.

As a result, binding was not confirmed in the control (NCI-H292 MOCK) regardless of the concentration of the WA-A1-3389 antibody. On the other hand, in human lung cancer cells in which IGSF1 is overexpressed (NCI-H292 IGSF1 O/E), the EC50 value of the WM-A1-3389 antibody was confirmed to be about 69 nM (FIG. 6).

### Example 4. Analysis of Antigen Specificity of Anti-IGSF1 Antibody for IGSF1 Antigen in Cells

In order to analyze the antigen specific binding capacity (target selectivity) of the WM-A1-3389 antibody to the IGSF1 antigen at a cellular level, binding of the WM-A1-3389 antibody to IGSF1 expressed in cells was confirmed using human lung cancer cells in which IGSF1 is overexpressed (NCI-H292 IGSF1 O/E).

Specifically, the medium of human lung cancer cells in which IGSF1 is overexpressed (NCI-H292 IGSF1 O/E) and a control thereof (NCI-H292 MOCK) was removed, washed once with PBS, and then treated with 2 ml of 0.25% trypsin-EDTA, and the cells were isolated. The isolated cells were diluted with 8 ml of PBS containing 2% FBS and 0.05% sodium azide (hereinafter referred to as FACS buffer), and then the supernatant was removed by centrifugation at 1,200 rpm for 1 minute. Then, the cells were resuspended in FACS buffer to 1×10⁵ cells/ml. Thereafter, 1 ml of each was dispensed in a FACS tube, and the supernatant was removed by centrifugation at 1,200 rpm for 1 minute.

The pellet remaining in the FACS tube was released by vortexing, and 0.4 μg of the human IgG isotype antibody (Bio X cell, BE0297) or the WM-A1-3389 antibody was added per 200 μl of FACS buffer, and then reacted at 4° C. for 30 minutes. After the reaction was completed, 1 ml of FACS buffer was added to each tube, and the supernatant was removed by centrifugation at 1,200 rpm for 1 minute. This process was performed a total of two times. The cell pellet remaining in the FACS tube was released by vortexing, and 0.4 μg of FITC-labeled goat anti-human IgG antibody (Invitrogen, 62-8411) was added per 200 μl of FACS buffer and reacted while blocking the light at 4° C. for 30 minutes.

After the reaction was completed, 1 ml of FACS buffer was added to each tube, and the supernatant was removed by centrifugation at 1,200 rpm for 1 minute. This process was performed a total of two times. Finally after removal of the supernatant, the remaining pellet was resuspended in 200 μl of FACS buffer and analyzed by FACS. For FACS analysis, FITC fluorescence value labeled in each cell was measured using a BD LSRFortessa™ flow cytometer, and the results were analyzed using a FlowJo software. In this case, NCI-H292 MOCK cells were used as a control for IGSF1 overexpressing cells, and human IgG isotype was used as a control for the WM-A1-3389 antibody.

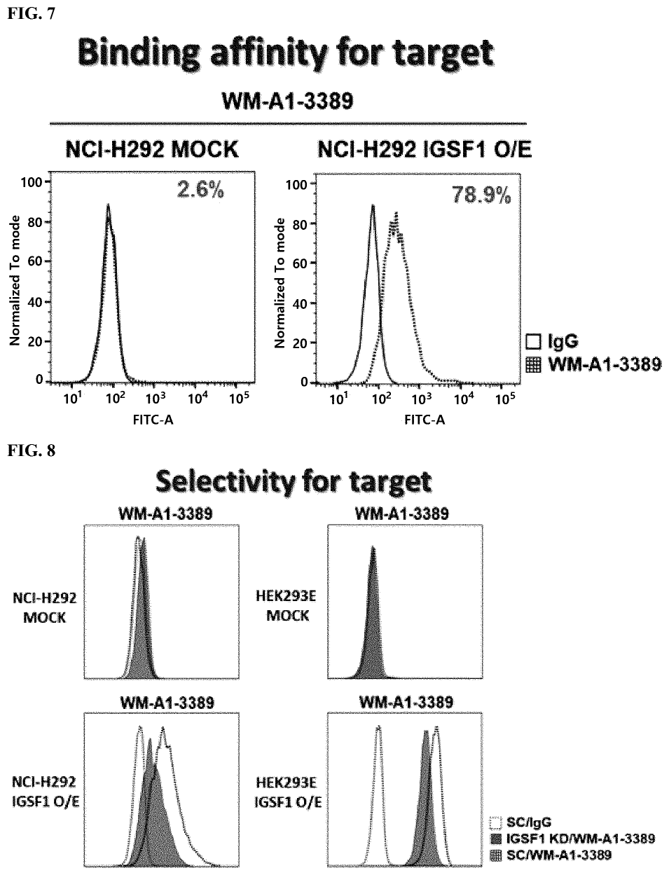

As a result, the group treated with the WM-A1-3389 antibody showed a binding capacity of about 2.6% in the control (NCI-H292 MOCK) and a binding capacity of about 78.9% in IGSF1 overexpressing cells (NCI-H292 IGSF1 O/E) compared to the group treated with the IgG isotype (FIG. 7).

Next, NCI-H292 IGSF1 O/E cells and HEK293E IGSF1 O/E cells in which IGSF1 is overexpressed were transfected with shRNA (hereinafter referred to as shIGSF1) that specifically binds to mRNA encoding IGSF1 to reduce the expression of IGSF1 (hereinafter referred to as IGSF1 K/D cells), and then the binding capacity of the WM-A1-3389 antibody to the IGSF1 antigen in the cells was measured. In this case, scramble RNA without shIGSF1 (hereinafter referred to as sc cell) was used as a control of transfection (IGSF1 K/D), and the human IgG isotype was used as a control for the WM-A1-3389 antibody. The antigen specificity of the WM-A1-3389 antibody was compared to the binding capacity in IGSF1 K/D cells based on the binding capacity in sc cells. In addition, NCI-H292 MOCK cells and HEK293E MOCK cells were used as controls for IGSF1 overexpressing cells, respectively.

Specifically, the media of NCI-H292 (IGSF1 O/E and MOCK) and HEK293E (IGSF1 O/E and MOCK) cell lines were removed and washed once with PBS, and then NCI-H292 cells were treated with 2 ml of 0.25% trypsin-EDTA, and HEK293E cells were treated with 2 ml of 0.05% trypsin-EDTA, respectively, and the cells were isolated. The isolated cells were diluted with 8 ml of culture medium, and then the supernatant was removed by centrifugation at 800 rpm for 3 minutes. The remaining cells were resuspended to a concentration of 1×10⁵ cells/ml (NCI-H292) and 0.5×10⁵ cells/ml (HEK293E), respectively, and then 3 ml of the cells was added to a 60 mm culture plate, and cultured in a cell incubator at 37° C. for one day. The next day, shIGSF1 transfection was performed. 200 μl of jet PRIME buffer and 10 nM of shIGSF1 were added and mixed in a 1.5 ml tube, and then 4 μl of jet PRIME reagent was added, mixed, and reacted at room temperature for 10 minutes. Then, the medium of the cells prepared the day before was replaced, and then 200 μl of the transfection mixture was added to each cell and reacted in a cell incubator for 24 hours. After 24 hours, it was replaced with a fresh culture medium and further cultured for 24 hours.

For the transfected cells, the medium was removed, and FACS analysis was performed in the same manner as above.

As a result, the binding of the WM-A1-3389 antibody compared to the group treated with the human IgG isotype was confirmed in the sc cell line. In addition, when the IGSF1 expression was reduced based on the binding capacity (IGSF1 K/D cells), it was confirmed that the binding capacity of the WM-A1-3389 antibody was reduced together (FIG. 8).

### Example 5. Analysis of Immuno Anticancer Efficacy of Anti-IGSF1 Antibody in Lung Cancer Cell Spheroids

In order to analyze the immuno anticancer efficacy of the WM-A1-3389 antibody at a cellular level, lung cancer cell spheroids and peripheral blood mononuclear cells (PBMC) were co-cultured to confirm death of tumor-infiltrating lymphocytes (TIL) and immunogenic cell.

Co-culture of lung cancer cell spheroids and peripheral blood mononuclear cells was performed in the same manner as in Example 2.2.

The co-cultured cells and the supernatant were collected in a tube, and the supernatant was removed by centrifugation at 1,200 rpm for 2 minutes. The cell pellet was made into single cells by treatment with 500 μl of 0.25% trypsin-EDTA and then diluted with 2 ml of PBS containing 2% FBS and 0.05% NaN₃ (hereinafter referred to as FACS buffer), and then the supernatant was removed by centrifugation at 1,200 rpm for 3 minutes. The remaining cell pellet was resuspended in 200 μl of FACS buffer, and then anti-HMGB1 antibody (Biolegend, 651408) and anti-Hsp90 antibody (Enzo Life Science, ADI-SPA-830PE-D) were added and stained at 4° C. for 30 minutes.

1 ml of FACS buffer was added to each tube, and the supernatant was removed by centrifugation at 1,200 rpm for 2 minutes. This process was repeated a total of two times. Thereafter, analysis was performed using a BD LSRFortessa™ flow cytometer. The results of FACS analysis were analyzed using a FlowJo software. In addition, tumor-infiltrating lymphocytes (TIL) were observed under a fluorescence microscope. In this case, the human IgG isotype was used as a control for the WM-A1-3389 antibody.

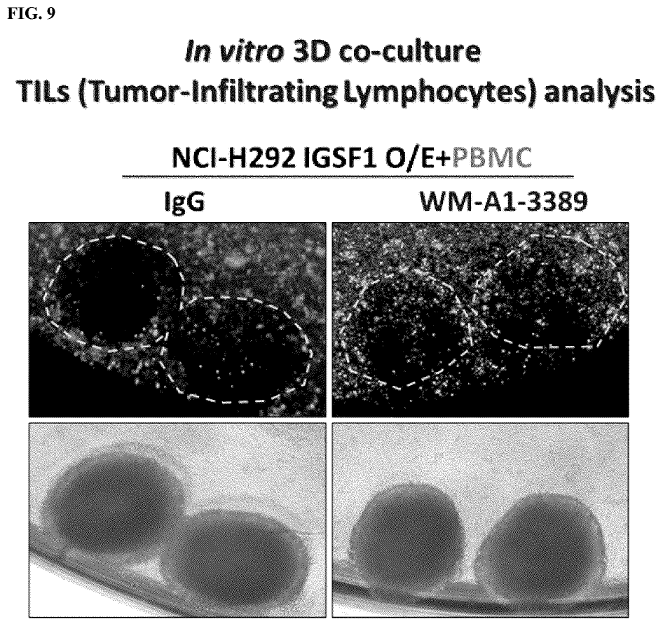

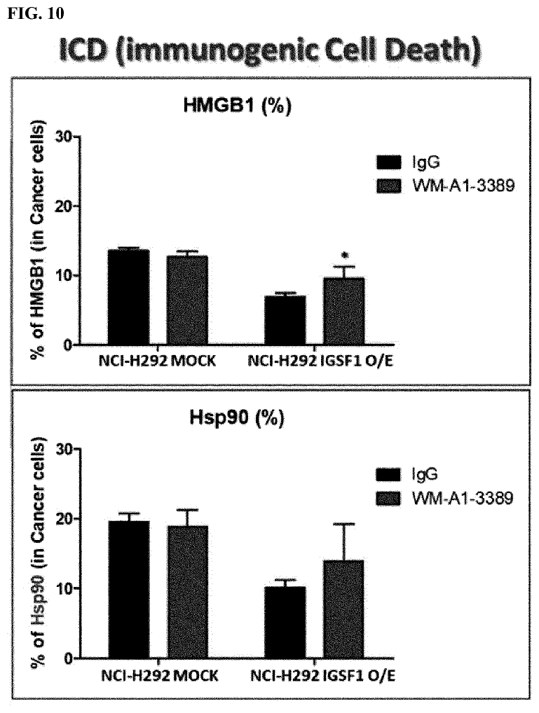

As a result, it was confirmed that tumor-infiltrating lymphocytes (TIL) were increased in the group treated with the WM-A1-3389 antibody compared to the control in IGSF1 overexpressing lung cancer cell (NCI-H292 IGSF1 O/E) spheroids (FIG. 9). In addition, it was confirmed that immunogenic cell death (ICD) was also increased in the group treated with the WM-A1-3389 antibody compared to the control in IGSF1 overexpressing lung cancer cell spheroids (FIG. 10).

### Example 6. Analysis of Tumor Growth Inhibition Efficacy of Anti-IGSF1 Antibody in Allograft Mouse Model

In order to confirm the anticancer efficacy of the WM-A1-3389 antibody at an animal level, peripheral blood mononuclear cell humanized model (PBMC humanized model) mice were transplanted with human lung cancer cells in which IGSF1 is overexpressed (NCI-H292 IGSF1 O/E), and then the tumor growth inhibition efficacy of the WM-A1-3389 antibody was evaluated.

Specifically, 6-week-old female peripheral blood mononuclear cell humanized mice (Gem biosciences) were purchased and acclimatized for 1 week, and then IGSF1 overexpressing human lung cancer cells NCI-H292 IGSF1 O/E (5×10⁶ cells/animal) were diluted in PBS and Matrigel and injected subcutaneously (200 μl) into the right dorsal side of the mice. When the tumor size reached about 120 mm³, IgG isotype (control) or the WM-A1-3389 antibody was administered intraperitoneally at a dose of 10 mg/kg, respectively. Administration was performed once every 3 days for 4 weeks, and the tumor size and body weight of the mice were measured twice a week. On day 22 of administration, blood and tumors from the satellite group mice were obtained and subjected to FACS analysis. After the administration was completed, the experimental animals were euthanized, the tumors were extracted, and the weight was measured. The human IgG isotype was used as a control for the WM-A1-3389 antibody.

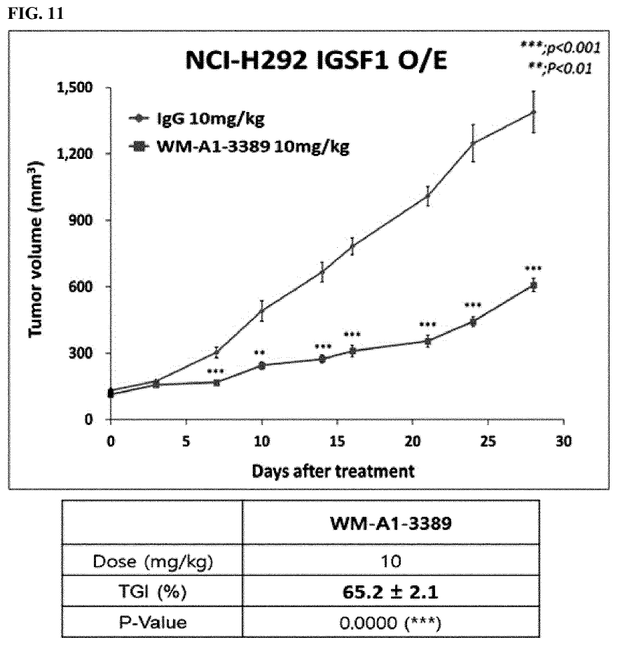

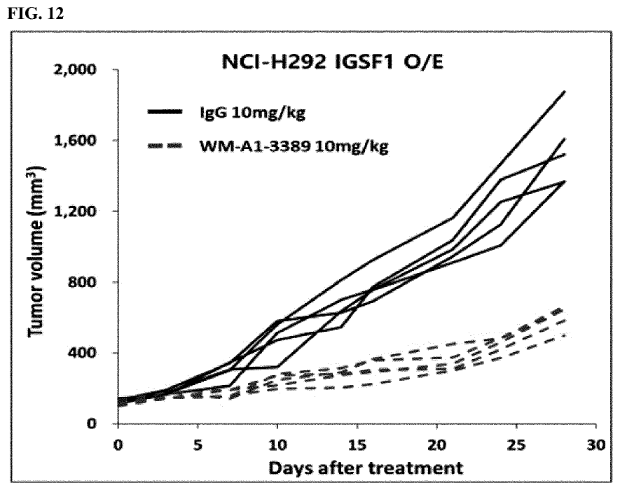

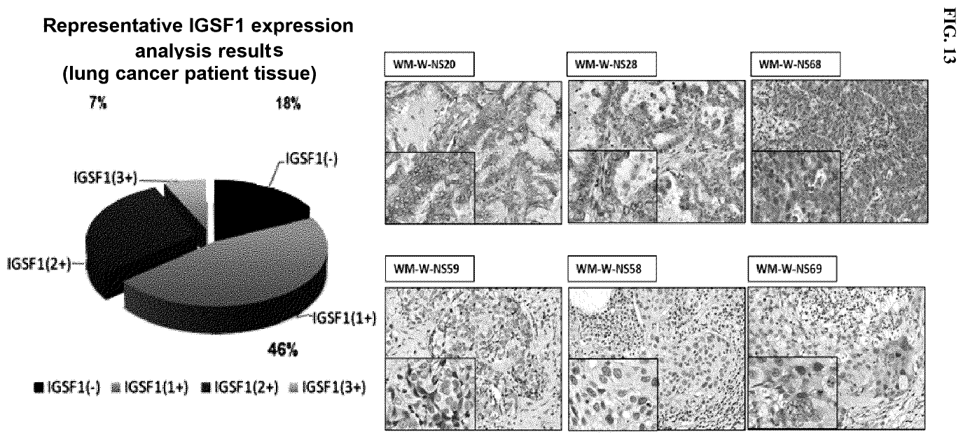

As a result, the group administered with the WM-A1-3389 antibody exhibited high tumor growth inhibition efficacy compared to the control, and exhibited a tumor inhibition rate (TGI) of about 64.5% (FIG. 11). In addition, it was confirmed that tumor growth was inhibited even for individual subjects (FIG. 12).

### Example 7. Analysis of IGSF1 Expression in Caucasian Lung Cancer Patient Tissue

The expression of IGSF1 in Caucasian lung cancer patient tissue was confirmed by immunohistochemistry staining method.

Specifically, a section of the tissue of the human non-small cell lung cancer patient was deparaffinized and rehydrated, and then soaked in a target repair buffer, and then heated in a microwave for 15 minutes for heat-induced epitope repair. Thereafter, it was reacted in a target repair buffer for another 30 minutes. Thereafter, it was washed 3 times with Tris buffered saline with 0.05% Tween 20 (TBS-T), and then blocked with a blocking solution for 60 minutes. The primary antibody was an anti-IGSF1 antibody (Santacruz, sc-393786), which was 1:100 diluted and allowed to be bound overnight at 4° C. The next day, the tissue section was washed 3 times with TBS-T and then reacted with an endogenous peroxidase blocking reagent (Cell Marque, 925B) for 5 minutes. Then, the secondary antibody (Vector, PK-6101 PK-6102) was allowed to be bound at room temperature for 60 minutes. Thereafter, it was washed 3 times with TBS-T, treated with avidin-biotin, and reacted for 60 minutes, and then the DAB staining (Vector, SK-4100) was performed. The stained tissue section was observed under a microscope (FIG. 13).

1-12. (canceled)

13. An antibody specific for IGSF1 or a fragment thereof, comprising
a heavy chain variable region comprising H-CDR1 of SEQ ID NO: 1, H-CDR2 of SEQ ID NO: 2 and H-CDR3 of SEQ ID NO: 3; and
a light chain variable region comprising L-CDR1 of SEQ ID NO: 4, L-CDR2 of SEQ ID NO: 5 and L-CDR3 of SEQ ID NO: 6.

14. The antibody specific for IGSF1 or fragment thereof according to claim 13, wherein
the heavy chain variable region has the amino acid sequence of SEQ ID NO: 7; and
the light chain variable region has the amino acid sequence of SEQ ID NO: 8.

15. A polynucleotide encoding a heavy chain variable region comprising H-CDR1 of SEQ ID NO: 1, H-CDR2 of SEQ ID NO: 2 and H-CDR3 of SEQ ID NO: 3.

16. A polynucleotide encoding a light chain variable region comprising L-CDR1 of SEQ ID NO: 4, L-CDR2 of SEQ ID NO: 5 and L-CDR3 of SEQ ID NO: 6.

17. An expression vector comprising the polynucleotide according to claim 15.

18. An expression vector comprising the polynucleotide according to claim 16.

19. A transformed cell into which the expression vector according to claim 17 is introduced.

20. A transformed cell into which the expression vector according to claim 18 is introduced.

21. A method of producing an antibody specific for IGSF1 or a fragment thereof, comprising:
i) culturing the transformed cell according to claim 19; and
ii) recovering the antibody specific for IGSF1 or the fragment thereof.

22. A method of producing an antibody specific for IGSF1 or a fragment thereof, comprising:
i) culturing the transformed cell according to claim 20; and
ii) recovering the antibody specific for IGSF1 or the fragment thereof.

23. A method for preventing or treating cancer, the method comprising administering to a subject the antibody specific for IGSF1 or fragment thereof according to claim 13.

24. The method according to claim 23, wherein the cancer is one in which IGSF1 is overexpressed.

25. The method according to claim 23, wherein the cancer is any one selected from the group consisting of gastric cancer, liver cancer, lung cancer, non-small cell lung cancer, colorectal cancer, bladder cancer, bone cancer, blood cancer, breast cancer, melanoma, thyroid cancer, parathyroid cancer, bone marrow cancer, rectal cancer, throat cancer, laryngeal cancer, esophageal cancer, pancreatic cancer, tongue cancer, skin cancer, brain tumor, uterine cancer, head or neck cancer, gallbladder cancer, oral cancer, perianal cancer, colon cancer, and central nervous system tumor.

26. A pharmaceutical composition comprising the antibody specific for IGSF1 or fragment thereof according to claim 13 as an active ingredient.


SEQUENCE LISTING (24 sequences)

SEQ ID NO: 1 - protein, 8 aa, Artificial Sequence
  H-CDR1 of WM-A1-3389
GGTFSTYA

SEQ ID NO: 2 - protein, 8 aa, Artificial Sequence
  H-CDR2 of WM-A1-3389
IIPFVGTV

SEQ ID NO: 3 - protein, 11 aa, Artificial Sequence
  H-CDR3 of WM-A1-3389
VRDGGRSYFDS

SEQ ID NO: 4 - protein, 8 aa, Artificial Sequence
  L-CDR1 of WM-A1-3389
TSNIGSNL

SEQ ID NO: 5 - protein, 3 aa, Artificial Sequence
  L-CDR2 of WM-A1-3389
DNH

SEQ ID NO: 6 - protein, 11 aa, Artificial Sequence
  L-CDR3 of WM-A1-3389
VAWDDSLNGYV

SEQ ID NO: 7 - protein, 118 aa, Artificial Sequence
  Variable region of heavy chain of WM-A1-3389
QVQLVQSGAEVKRPGSSVKVSCKASGGTFSTYAISWVRQAPGQGLEWMGRIIPFVGTVDYAQKFQDRVTITADKSTNTAYMELSSLRSEDTAVYYCVRDGGRSYFDSWGPGILVTVSS

SEQ ID NO: 8 - protein, 110 aa, Artificial Sequence
  Variable region of light chain of WM-A1-3389
QFVLTQPPSVSAAPGQDVIISCSGNTSNIGSNLVSWFQQFPETAPKLLIYDNHKRPSGISDRFSGTKSGTSASLAISGLQSEDEADYYCVAWDDSLNGYVFGTGTKVTVL

SEQ ID NO: 9 - DNA, 354 bp, Artificial Sequence
  Polynucleotide coding variable region of heavy chain of WM-A1-3389
caggtgcagctggtacagtctggggctgaggtgaagaggcctgggtcctcggtgaaggtctcctgcaaggcttctggaggcaccttcagcacctatgctatcagctgggtgcgacaggcccctggacaagggcttgagtggatgggaaggatcatccctttcgttggtacagtagactacgcacagaagttccaggacagagtcacaattaccgcggacaaatccacgaacacagcctacatggagctgagcagcctgagatctgaggacacggccgtgtattactgtgtgagagatgggggccgtagttattttgactcctggggcccgggaatcctggtcaccgtctcctca

SEQ ID NO: 10 - DNA, 330 bp, Artificial Sequence
  Polynucleotide coding variable region of light chain of WM-A1-3389
cagttcgtgctgactcagccgccctcagtctctgcggccccagggcaggacgtcatcatctcttgctctggaaacacttccaacattgggagtaaccttgtctcctggttccagcaattcccagagacagcccccaaactcctgatttatgacaatcataagcgaccctcaggaatttctgaccgattctctggcaccaagtctggtacctcagcctccctggccatcagtgggctccagtctgaggatgaggctgattattactgtgtggcatgggatgacagtctgaatggttatgtcttcggaactgggaccaaggtcaccgtccta

SEQ ID NO: 11 - protein, 25 aa, Artificial Sequence
  H-FR1 of WM-A1-3389
QVQLVQSGAEVKRPGSSVKVSCKAS

SEQ ID NO: 12 - protein, 17 aa, Artificial Sequence
  H-FR2 of WM-A1-3389
ISWVRQAPGQGLEWMGR

SEQ ID NO: 13 - protein, 38 aa, Artificial Sequence
  H-FR3 of WM-A1-3389
DYAQKFQDRVTITADKSTNTAYMELSSLRSEDTAVYYC

SEQ ID NO: 14 - protein, 11 aa, Artificial Sequence
  H-FR4 of WM-A1-3389
WGPGILVTVSS

SEQ ID NO: 15 - protein, 25 aa, Artificial Sequence
  L-FR1 of WM-A1-3389
QFVLTQPPSVSAAPGQDVIISCSGN

SEQ ID NO: 16 - protein, 17 aa, Artificial Sequence
  L-FR2 of WM-A1-3389
VSWFQQFPETAPKLLIY

SEQ ID NO: 17 - protein, 36 aa, Artificial Sequence
  L-FR3 of WM-A1-3389
KRPSGISDRFSGTKSGTSASLAISGLQSEDEADYYC

SEQ ID NO: 18 - protein, 10 aa, Artificial Sequence
  L-FR4 of WM-A1-3389
FGTGTKVTVL

SEQ ID NO: 19 - protein, 756 aa, Artificial Sequence
  IGSF1
EETEIVMPTPKPELWAETNFPLAPWKNLTLWCRSPSGSTKEFVLLKDGTGWIATRPASEQVRAAFPLGALTQSHTGSYHCHSWEEMAVSEPSEALELVGTDILPKPVISASPTIRGQELQLRCKGWLAGMGFALYKEGEQEPVQQLGAVGREAFFTIQRMEDKDEGNYSCRTHTEKRPFKWSEPSEPLELVIKEMYPKPFFKTWASPVVTPGARVTFNCSTPHQHMSFILYKDGSEIASSDRSWASPGASAAHFLIISVGIGDGGNYSCRYYDFSIWSEPSDPVELVVTEFYPKPTLLAQPGPVVFPGKSVILRCQGTFQGMRFALLQEGAHVPLQFRSVSGNSADFLLHTVGAEDSGNYSCIYYETTMSNRGSYLSMPLMIWVTDTFPKPWLFAEPSSVVPMGQNVTLWCRGPVHGVGYILHKEGEATSMQLWGSTSNDGAFPITNISGTSMGRYSCCYHPDWTSSIKIQPSNTLELLVTGLLPKPSLLAQPGPMVAPGENMTLQCQGELPDSTFVLLKEGAQEPLEQQRPSGYRADFWMPAVRGEDSGIYSCVYYLDSTPFAASNHSDSLEIWVTDKPPKPSLSAWPSTMFKLGKDITLQCRGPLPGVEFVLEHDGEEAPQQFSEDGDFVINNVEGKGIGNYSCSYRLQAYPDIWSEPSDPLELVGAAGPVAQECTVGNIVRSSLIVVVVVALGVVLAIEWKKWPRLRTRGSETDGRDQTIALEECNQEGEPGTPANSPSSTSQRISVELPVPI

SEQ ID NO: 20 - protein, 784 aa, Artificial Sequence
  IGSF1 with leader sequence and His tag
MGWSYIILFLVATAADVHSQHHHHHHHHEETEIVMPTPKPELWAETNFPLAPWKNLTLWCRSPSGSTKEFVLLKDGTGWIATRPASEQVRAAFPLGALTQSHTGSYHCHSWEEMAVSEPSEALELVGTDILPKPVISASPTIRGQELQLRCKGWLAGMGFALYKEGEQEPVQQLGAVGREAFFTIQRMEDKDEGNYSCRTHTEKRPFKWSEPSEPLELVIKEMYPKPFFKTWASPVVTPGARVTFNCSTPHQHMSFILYKDGSEIASSDRSWASPGASAAHFLIISVGIGDGGNYSCRYYDFSIWSEPSDPVELVVTEFYPKPTLLAQPGPVVFPGKSVILRCQGTFQGMRFALLQEGAHVPLQFRSVSGNSADFLLHTVGAEDSGNYSCIYYETTMSNRGSYLSMPLMIWVTDTFPKPWLFAEPSSVVPMGQNVTLWCRGPVHGVGYILHKEGEATSMQLWGSTSNDGAFPITNISGTSMGRYSCCYHPDWTSSIKIQPSNTLELLVTGLLPKPSLLAQPGPMVAPGENMTLQCQGELPDSTFVLLKEGAQEPLEQQRPSGYRADFWMPAVRGEDSGIYSCVYYLDSTPFAASNHSDSLEIWVTDKPPKPSLSAWPSTMFKLGKDITLQCRGPLPGVEFVLEHDGEEAPQQFSEDGDFVINNVEGKGIGNYSCSYRLQAYPDIWSEPSDPLELVGAAGPVAQECTVGNIVRSSLIVVVVVALGVVLAIEWKKWPRLRTRGSETDGRDQTIALEECNQEGEPGTPANSPSSTSQRISVELPVPI

SEQ ID NO: 21 - protein, 448 aa, Artificial Sequence
  Heavy chain of WM-A1-3389
QVQLVQSGAEVKRPGSSVKVSCKASGGTFSTYAISWVRQAPGQGLEWMGRIIPFVGTVDYAQKFQDRVTITADKSTNTAYMELSSLRSEDTAVYYCVRDGGRSYFDSWGPGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 217 aa, Artificial Sequence
  Light chain of WM-A1-3389
QFVLTQPPSVSAAPGQDVIISCSGNTSNIGSNLVSWFQQFPETAPKLLIYDNHKRPSGISDRFSGTKSGTSASLAISGLQSEDEADYYCVAWDDSLNGYVFGTGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1344 bp, Artificial Sequence
  Polynucleotide coding heavy chain of WM-A1-3389
caagttcagctggttcagtctggcgccgaagtgaaaagacctggcagcagcgtgaaggtgtcctgcaaagcttctggcggcaccttcagcacctacgccatctcttgggttcgacaggcccctggacaaggcctggaatggatgggcagaatcatcccctttgtgggcaccgtggactacgcccagaaattccaggacagagtgaccatcaccgccgacaagagcaccaacaccgcctacatggaactgagcagcctgagaagcgaggacaccgccgtgtactactgcgtccgagatggcggcagaagctacttcgattcttggggccctggcatcctggtcacagtgtctagcgcctctacaaagggccccagcgttttcccactggctcctagcagcaagagcacaagcggaggaacagccgctctgggctgtctggtcaaggactactttcccgagcctgtgaccgtgtcttggaactctggcgctctgacaagcggcgtgcacacatttccagccgtgctgcaaagcagcggcctgtactctctgagcagcgtcgtgacagtgccaagcagctctctgggcacccagacctacatctgcaatgtgaaccacaagcctagcaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggacccagaagtgaagttcaattggtacgtggacggcgtggaagtgcacaacgccaagaccaagcctagagaggaacagtacaactccacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgacaaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgaccgtggacaagtccagatggcagcagggcaacgtgttcagctgcagcgtgatgcacgaggccctgcacaaccactacacacagaagtccctgtctctgagccccggcaaa

SEQ ID NO: 24 - DNA, 651 bp, Artificial Sequence
  Polynucleotide coding light chain of WM-A1-3389
cagtttgttctgacacagcctccaagcgtgtccgccgctcctggacaggatgtgatcatcagctgcagcggcaacaccagcaacatcggcagcaatctggtgtcctggttccagcagttccccgagacagcccctaagctgctgatctacgacaaccacaagcggcccagcggcatcagcgatagattcagcggcacaaagagcggcaccagcgcttctctggccatctctggactgcagagcgaggacgaggccgactactattgtgtggcctgggacgacagcctgaacggctatgtgtttggcaccggcaccaaagtgaccgtgctgagaacagtggccgctcctagcgtgttcatcttcccaccttccgacgagcagctgaagtctggcacagccagcgttgtgtgcctgctgaacaacttctaccctcgggaagccaaggtgcagtggaaggtggacaatgccctgcagtccggcaacagccaagagagcgtgacagagcaggacagcaaggactccacctacagcctgagcagcaccctgacactgagcaaggccgattacgagaagcacaaggtgtacgcctgcgaagtgacacaccagggcctgtctagccctgtgaccaagagcttcaacagaggcgagtgc